(19) United States
(12) Patent Application Publication
(10) Pub. No.: US 2018/0228497 A1
(43) Pub. Date: Aug. 16, 2018

(54) TOURNIQUET

(71) Applicants: Michael J. DIMINO, Freehold, NJ (US); Michael C. DIMINO, Freehold, NJ (US); Alfonse DIMINO, Freehold, NJ (US)

(72) Inventors: Michael J. DIMINO, Freehold, NJ (US); Michael C. DIMINO, Freehold, NJ (US); Alfonse DIMINO, Freehold, NJ (US)

(21) Appl. No.: 15/932,437

(22) Filed: May 22, 2017

Related U.S. Application Data
(60) Provisional application No. 62/496,016, filed on Oct. 3, 2016.
(60) Provisional application No. 62/496,017, filed on Oct. 3, 2016.
(60) Provisional application No. 62/496,018, filed on Oct. 3, 2016.

(51) Int. Cl. A61B 17/132; A61F 15/00

(52) U.S. Cl. CPC A61B 17/1327; A61F 15/006

(57) ABSTRACT

A tourniquet with two locking, two release and two tension mechanisms that occludes blood flow in extremities, but can also be used as a pressure strap to hold gauze over wounds on different part of the body including torso.

### CROSS-REFERENCE TO RELATED APPLICATIONS

This application claims benefit under 35 U.S.C. § 119(e) to Provisional Application Ser. Nos. 62/496,016, filed on Oct. 3, 2016; 62/496,017, filed on Oct. 3, 2016; and 62/496,018, filed on Oct. 3, 2016, the disclosures of which are incorporated herein by reference in their entireties.

### BACKGROUND OF THE INVENTION

The present invention is directed to tourniquets, and, more particularly, to tourniquets with a windlass tightening mechanism and other accessories.

We now live in a post 911 environment in a World where violent threats and terrorist attacks are literally knocking at our door; school mass shooting, bombings, vehicle attacks is the reality of today. These attacks cause trauma wounds and hemorrhaging to the extremities and in some cases loss of limb if not immediately treated, the victim will die from blood loss within 90 seconds to 5 minutes.

The White House and Homeland Security has begun a campaign called “Stop the Bleed” it calls for Worldwide distribution of tourniquets to be placed in school, vehicles, first responders, office buildings, airports, stadiums, outdoor events, all law enforcement and everywhere there are first aid kits and defibrillators.

The current tourniquet in use today require prior training in order to apply properly, the current application time is at least 5 minutes, a tourniquet is needed that anyone can apply within a few seconds, a tourniquet that a bystander can apply to a victim without prior training, because no matter how fast a first responder arrives, the bystander will always be first on the scene. The current invention is the first tourniquet that can be applied within seconds; its familiar design similar to a zip tie, allows almost anyone to be able to apply it.

Its water, oil and temperature resistant, it's been tested and approved by Homeland Security and first responders. It's also the first tourniquet that has an automatic timer that informs first responders how long the tourniquet was applied to help prevent tissue and muscle damage.

A windlass tightening mechanism is described in U.S. Pat. No. 7,842,067 see Esposito Nov. 30, 2010. In accordance with the present invention, Esposito tourniquet has an inner strap, that the windlass tension bar pulls in and tightens tourniquet.

Esposito also uses fabric material for the tourniquet strap FIG. 2 #14, line 26 “pull the inner strap” as other prior art, the tourniquet strap when made with Velcro which will fail if the Velcro becomes wet, dirty or is exposed to oil, also Velcro is not resistant to high heat or extreme cold. Rex McDonald U.S. Pat. No. 8,926,651B2 describes a Windlass Tourniquet that also uses Velcro as a strap.

### SUMMARY OF THE INVENTION

The present invention has the following features that make it unique:
Has a double locking and tension mechanism.
The S.T.A.T. Tourniquet is designed to interlock with 2 or more extra tourniquets to form one large tourniquet that can be used on the torso to hold pressure on gauze over wound forming a pressure strap.
S.T.A.T. Tourniquet pressure strap can support fractured hips, skull fractures, or to help occlude abdominal bleeding.
Can be worn on Molle compatible gear.
Self-activating liquid timing strip.
2 tension release levers.

### BRIEF DESCRIPTION OF THE DRAWING(S)

FIG. 1 is a perspective of a tourniquet in accordance with the present invention.

FIG. 2 shows one side of the strap component of the tourniquet of FIG. 1.

FIG. 3 is another perspective of the tourniquet of FIG. 1 with the leading edge folded over.

FIG. 4 is another perspective of the tourniquet of FIG. 1, showing its initial insertion into the head component.

FIG. 5 is a perspective of the windlass tensioning mechanism of the tourniquet of FIG. 1.

FIG. 6 shows a cross section through the head component of the tourniquet of FIG. 1.

FIG. 7 shows a side view of the tourniquet of FIG. 1.

FIG. 8 shows another perspective of the tourniquet of FIG. 1.

FIG. 9 shows a top view of the tourniquet of FIG. 1.

Other features and advantages of the present invention will become apparent from the following description of the invention which refers to the accompanying drawings.

### DETAILED DESCRIPTION OF EMBODIMENTS OF THE INVENTION

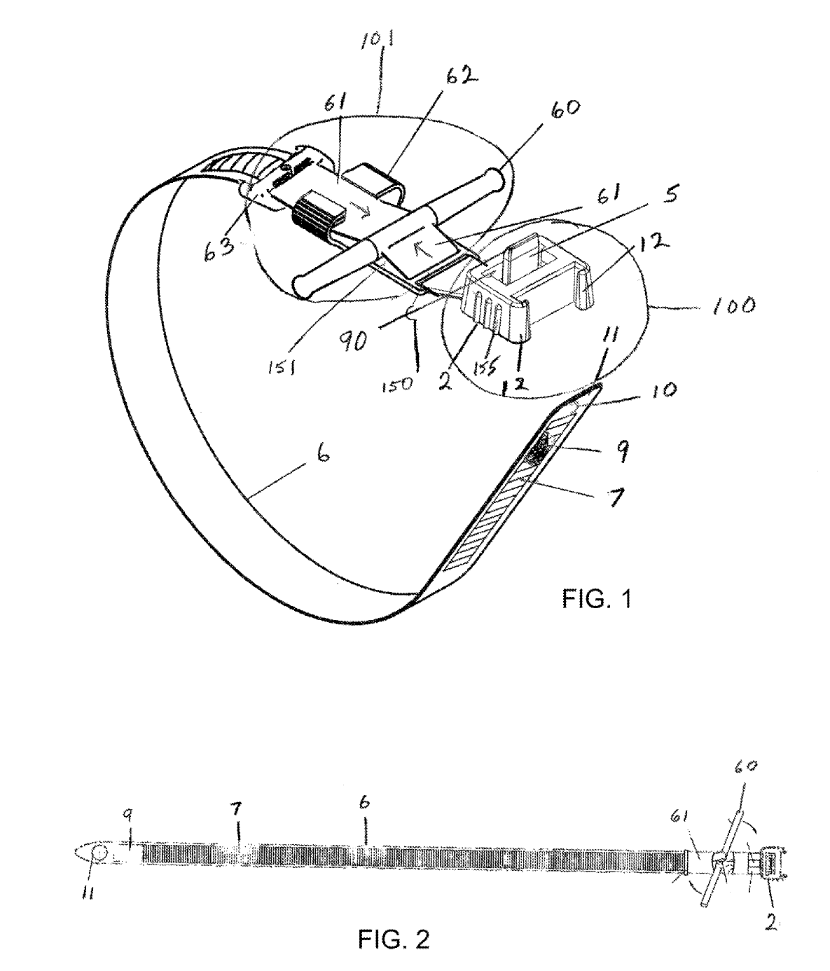

In FIG. 1:

#101 is the Windlass Locking and tension assembly;

#100 is the 2nd locking and tension adjustment head;

#6 is the serrated strap of the tourniquet;

#9 is the automatic timer;

#10 is the finger hole;

#11 is the tail of strap;

#63 is the buckle that attaches to the strap and plastic plate #151;

#61 is the fabric material that connects to Windlass rod #60;

#62 are the clips that hold Windlass rod #60;

#150 is the buckle that connects to #151 plastic plate and locking head #2;

#12 is a protruding mechanism that holds extra strap slack, it also serves as a safety cover over #5 release cover; and

#155 is the non-slip finger grips.

In FIG. 6:

#2 is the locking head;

#156 is the locking teeth that strap teeth interlock with;

#5 is the release lever that disengages locking teeth in the head with strap teeth #7;

#90 is where strap is inserted;

#71 keeps strap #6 straight, so strap evenly locks with teeth #156;

#12 is the protruding mechanism that holds extra slack of the strap;

#90 is where strap #11 is inserted; and

#14 sleeve that covers the Windlass locking mechanism.

FIG. 6 shows a side view of locking head #101.

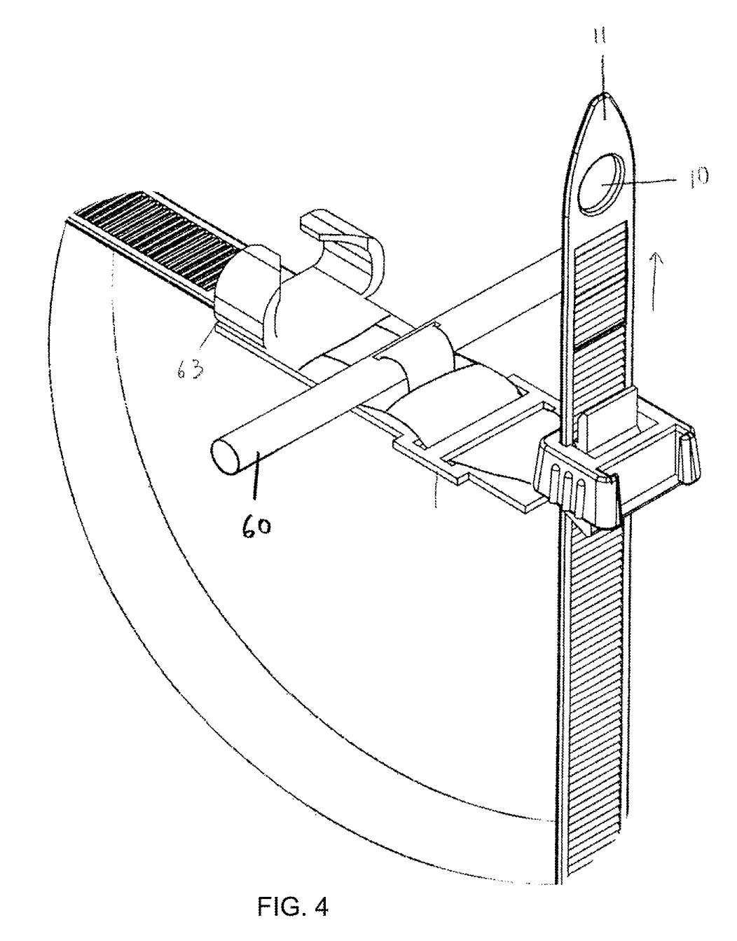

FIG. 4 shows strap #6 entering the locking head #2.

FIG. 1 shows the Windlass mechanism #101 and locking head #100 assembly.

FIG. 2 shows the complete tourniquet laid flat and direction of tension bar turning.

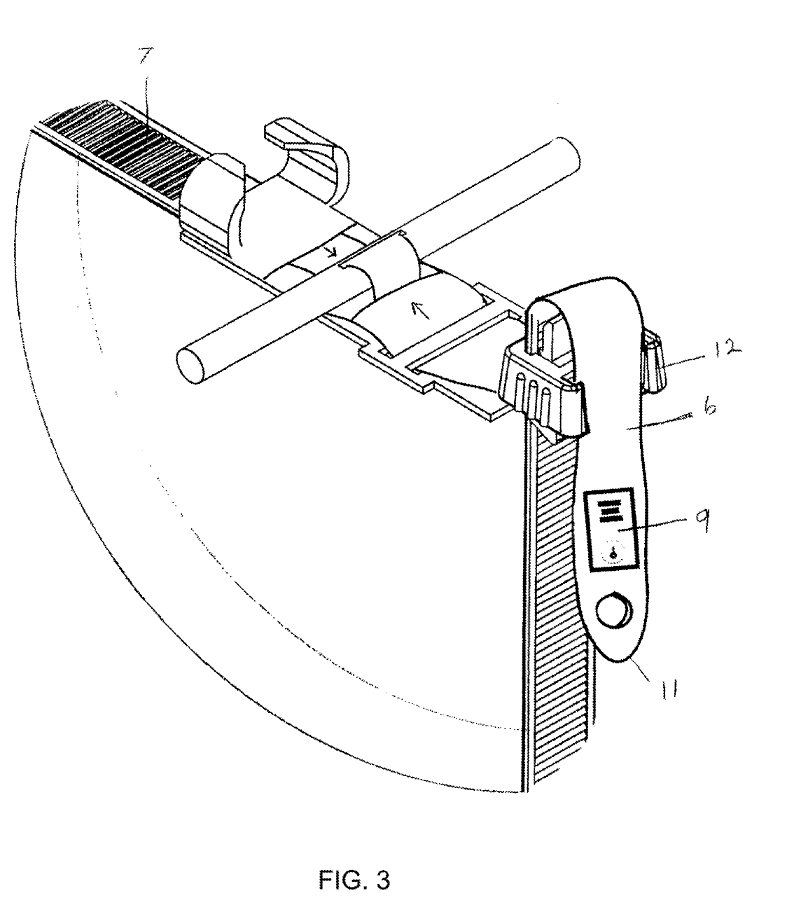

FIG. 3 shows strap #6 in slack holder #12, it also shows the timer #9.

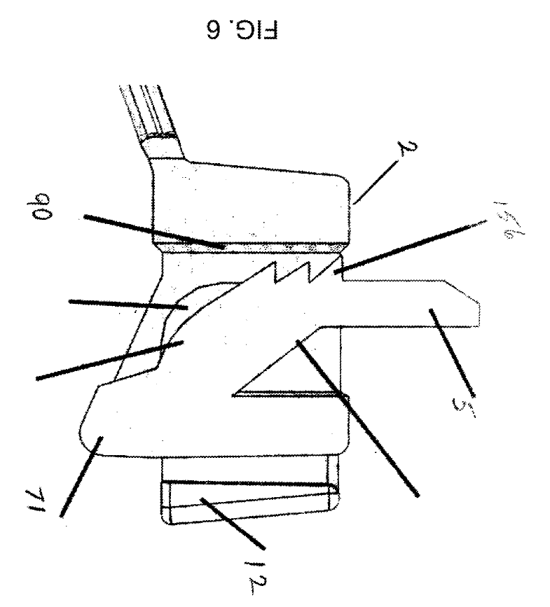

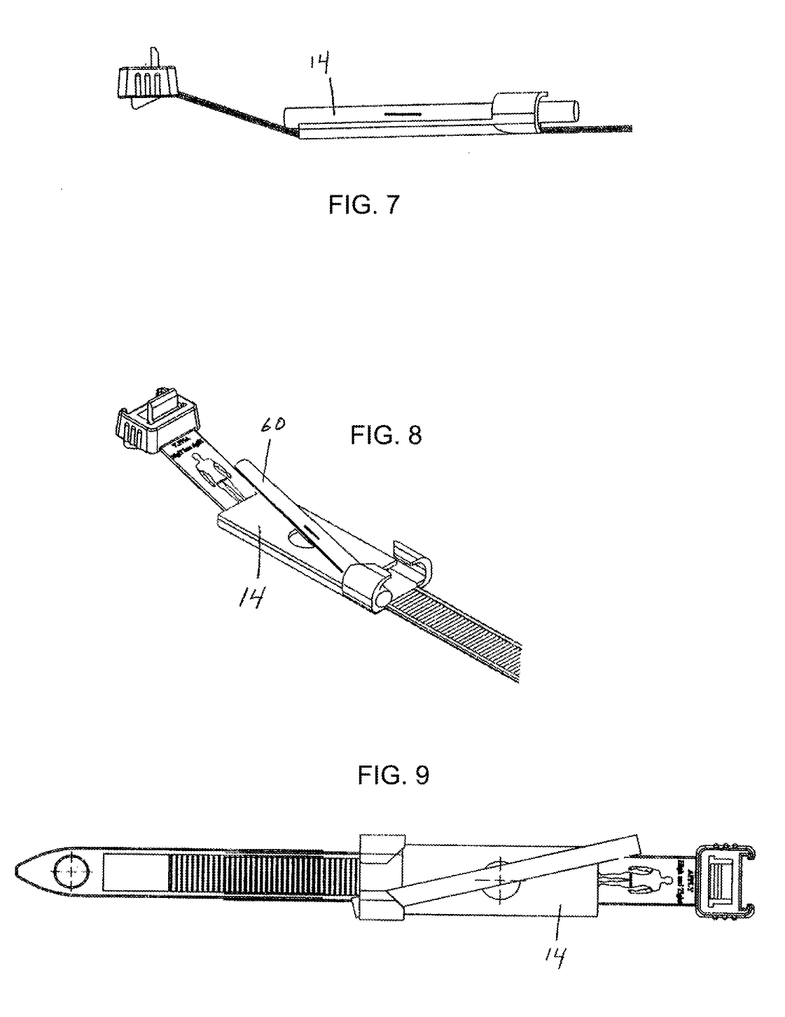

FIGS. 8, 9, and 7 show a different design tourniquet where there is a sleeve over windlass assembly #101.

FIG. 7 shows a side view of tourniquet with windlass sleeve #14.

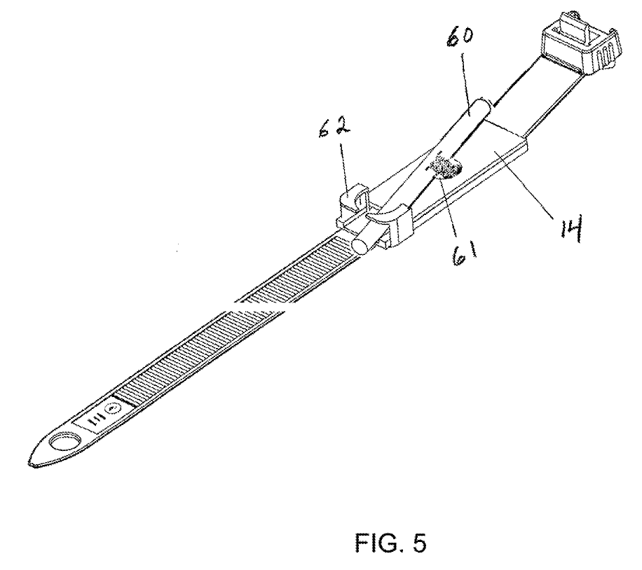

FIG. 5 shows tourniquet with windlass sleeve #14 and fabric material inserted into windlass tension rod.

The present invention eliminates the inner fabric and Velcro, and only tightens the plastic strap 6 FIG. 1. In addition to the windlass tension mechanism. The present invention has a second tension mechanism to tighten the tourniquet, it uses male teeth in head 90 FIG. 6 and interlocks with female teeth 7 on strap FIG. 1, to interlock with each other; female teeth on strap are only half of a millimeter spacing allow for gradual increase in tension by pulling strap away from head increases tension, tension adjustments in millimeters eliminating over tightening which causes pain, and in some cases the victim (If a child) can break bones. The tourniquet is also able to ratchet it down to fit the circumference of children, or the elderly.

In order to use the tourniquet, the strap is placed around the limb, the tail #11 of strap is inserted into #90 of locking head #2, the teeth #7 on strap interlock with teeth in the head #156 of FIG. 6. Extra slack of the strap is placed into holder #12; ifs also used as a safety cover for #5 the release lever.

In FIG. 3 to increase circumference tension of strap around the limb, tail #11 is pulled away from the head.

Should blood flow not stop then the Windlass rod #60 would be turned which would pull the fabric material #61 and strap #6 inward which would increase the tourniquet tension; once desired tension is accomplished, Windlass rod is stored in clip #62. In order to release tension wither the Windlass rod #60 is removed from clip #62 and unwound, or release lever #5 can be pulled backward to disengage the teeth on strap with teeth in the head.

According to the present invention, when tourniquet strap teeth engage with teeth in the head an audible sound can be heard to verify tourniquet has been properly applied.

According to the present invention, the locking head has a triple locking mechanism with teeth in the head that engage with teeth on the strap.

According to the present invention, the strap is made of a plastic material, which is flexible and can withstand temperatures of below 0 degrees and up to 250 degrees Fahrenheit.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

1.-23. (canceled)

24. A tourniquet with two locking, two release and two tension mechanisms that occludes blood flow in extremities, but can also be used as a pressure strap to hold gauze over wounds on different part of the body including torso.

25. The tourniquet of claim 24, including:
a first locking and tension mechanism comprising a strap that has serrated teeth all along the length of the strap;
a head that has an automatic locking mechanism in the form of teeth that interlock with teeth on the strap;
a second locking and tension mechanism comprising:
a tension bar, a buckle, and a windlass locking mechanism.

26. The tourniquet of claim 25, wherein the strap with serrated teeth is attached to a plastic plate that also has attached to it a buckle and clips that hold a windlass rod.

27. The tourniquet of claim 26, wherein the buckle is attached to another buckle by fabric material which then the fabric material is threaded into a plastic plate to give support to locking mechanism, the fabric material passes through the tension bar, and the other end of material is attached to another buckle that attaches to the locking head of the automatic locking mechanism.

28. The tourniquet of claim 27, wherein the fabric material is also attached to a tension bar when the tension bar is twisted it draws inward the fabric material and the strap causing more circumference pressure around the object or extremities which causes occlusion of blood flow, so that the more the tension bar is twisted the tighter the tourniquet becomes around the limb.

29. The tourniquet of claim 25, wherein attached to the buckle are two clip mechanisms to store tension rod.

30. The tourniquet of claim 24, wherein the tourniquet has a double locking mechanism first locking mechanism is the interlocking of strap teeth with the teeth in head, the second is a windlass mechanism, both are used to tighten tourniquet pressure around limb to occlude blood flow.

31. The tourniquet of claim 24, wherein the end of strap (tail) has a liquid timer strip; it automatically activated when reservoir of timer is ruptured when it passes through mouth of head, it can also be activated by manually pressing down on reservoir with finger, and the timer informs user the length of time the tourniquet has been on.

32. The tourniquet of claim 24, wherein the tourniquet can also be used as a tactical tool to bound limbs and carry equipment.

33. The tourniquet of claim 24, wherein the tourniquet is equipped with a body sensor that can read vital signs of body temperature, blood pressure, heart rate, and oxygen level, and the device can also detect if a victim is going into shock from loss of blood.

34. The tourniquet of claim 24, wherein the tourniquet is equipped with a transceiver and GPS receiver that can transmit victim's voice and vital signs to a remote location.

35. The tourniquet of claim 24, wherein the tourniquet strap is constructed so two tourniquets can be connected together to form one larger tourniquet.

36. The tourniquet of claim 24, wherein the strap has a chemical patch attached to it, which can be self-activated by the victim; the patch can be, but not limited to morphine lanacane etc. for relief of tourniquet pain or from wound.

37. The tourniquet of claim 24, the tourniquet has a visible light or infrared light so it can only be visible with infrared device.

38. The tourniquet of claim 24, wherein the tourniquet can be stored and packaged in a ready to deploy position, which allows the user to remove tourniquet from packaging using only one hand and slipping the tourniquet on to the limb and tightening, this is accomplished by storing tourniquet tail into mouth of locking head.

39. The tourniquet of claim 24, wherein the teeth on strap are distanced in millimeters which allows for slight tension adjustment, for partial or full occlusion, also eliminates over tightening that causes pain and possible broken bones.

40. The tourniquet of claim 24, wherein the material used for strap, is bullet, oil, dirt, water resistant and self-sealing.

41. The tourniquet of claim 24, wherein two tourniquets can be connected together to make one larger tourniquet to be used on torso area to hold pressure dressings or to hold in place fractured hip, to use as a junction tourniquet.

42. The tourniquet of claim 24, wherein built into the locking head mechanism is a one finger safety release lever to disengage locking teeth in head with teeth on strap and release of pressure.

43. The tourniquet of claim 24, wherein the tourniquet has a UV inhibitor.
